(19) United States
(12) Patent Application Publication
(10) Pub. No.: US 2025/0218559 A1
(43) Pub. Date: Jul. 3, 2025

(54) METHOD OF PROVIDING INTEGRATED REPORT FOR ENTERPRISE HEALTHCARE DATA

(71) Applicant: IRM Inc., Seoul (KR)

(72) Inventors: Seung Wook CHOI, Seongnam-si (KR); Min Ho LEE, Namyangju-si (KR)

(73) Assignee: IRM Inc., Seoul (KR)

(21) Appl. No.: 18/927,252

(22) Filed: Oct. 25, 2024

(30) Foreign Application Priority Data
Dec. 28, 2023 (KR) 10-2023-0193845
Feb. 28, 2024 (KR) 10-2024-0028445

(51) Int. Cl. G16H 15/00

(52) U.S. Cl. CPC G16H 15/00

(57) ABSTRACT

Provided is a method of providing an integrated report for enterprise healthcare data, the method including automatically identifying mapping regions between a first type report and a second type report different from the first type report, automatically generating an integrated report on the basis of the identified mapping regions, and adding at least some information included in non-mapping regions, which are regions of the first type report and the second type report other than the mapping regions, to the integrated report.

### CROSS-REFERENCE TO RELATED APPLICATION

This application claims priority to and the benefit of Korean Patent Application No. 10-2024-0028445, filed on Feb. 28, 2024 and Korean Patent Application No. 10-2023-0193845, filed on Dec. 28, 2023, the disclosure of which is incorporated herein by reference in its entirety.

### BACKGROUND

### 1. Field of the Invention

The present disclosure relates to a report provision method, and more particularly, to a method of providing a report for enterprise healthcare data.

### 2. Discussion of Related Art

The difference in the representation of medical information managed by different technologies has been a barrier to the implementation of big data-based healthcare services and has led to the necessity to integrate and manage medical information.

However, in the medical market, additional work is required in practice for mapping medical information directly, and various application programming interfaces (APIs) are being developed accordingly.

Therefore, in line with this trend, it is necessary to develop a deep learning-based mapping solution that is dynamically adaptable on the basis of an efficient mapping method and an actual usage environment.

### SUMMARY OF THE INVENTION

The present disclosure is directed to providing a method of providing an integrated report by merging different types of reports.

According to an aspect of the present disclosure, there is provided a method of providing an integrated report for enterprise healthcare data, the method including automatically identifying mapping regions between a first type report and a second type report different from the first type report, automatically generating an integrated report on the basis of the identified mapping regions, and adding information included in non-mapping regions, which are regions of the first type report and the second type report other than the mapping regions, to the integrated report.

The adding of the information to the integrated report may include determining relationships between field identification elements of the non-mapping regions and field identification elements of the mapping regions and identifying positions to which the non-mapping regions will be added in the integrated report on the basis of the relationships.

The determining of the relationships may include determining correlations between the non-mapping regions and the mapping regions on the basis of a previously generated integrated report.

The method may further include outputting at least one of a relation image and a relation report corresponding to the non-mapping regions when the non-mapping regions are a reference ratio or more.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a block diagram of an electronic device for generating an integrated report according to an exemplary embodiment of the present disclosure;

FIG. 2 is a flowchart illustrating a method of generating an integrated report according to an exemplary embodiment;

FIGS. 3A and 3B are diagrams of different types of reports according to an exemplary embodiment;

FIG. 4 is a flowchart illustrating a process of additionally searching a first type report and a second type report for mapping regions according to an exemplary embodiment; and

FIG. 5 is a diagram of a process in which an integrated report is generated by a plurality of trained models according to an exemplary embodiment.

### DETAILED DESCRIPTION OF EXEMPLARY EMBODIMENTS

Hereinafter, various embodiments of the present disclosure will be described with reference to the accompanying drawings. While the present disclosure can be modified in various ways and can have several embodiments, particular embodiments will be illustrated in the drawings and described in detail. However, it should be understood that the embodiments are not intended to limit various embodiments of the present disclosure to specific forms of implementation and include all modifications, equivalents, and/or substitutions within the spirit and technical scope of various embodiments of the present disclosure. In relation to description of drawings, like reference numbers are used for like components.

In various embodiments of the present disclosure, terms such as “include,” “have,” and the like indicate existence of features, numerals, steps, operations, components, parts, or combinations thereof stated herein and do not preclude existence or addition of one or more other features, numerals, steps operations, components, parts, or combinations thereof.

In various embodiments of the present disclosure, terms such as “and/or” and the like include any and all combinations of listed words. For example, “A and/or B” may include A, B, or both A and B.

In various embodiments of the present disclosure, terms such as “first,” “second,” and the like may be used to describe various components of various embodiments, but these terms do not limit the corresponding components. For example, the terms do not limit the order, importance, and/or the like of the corresponding components and may be used to distinguish one component from others.

When a component is referred to as being “connected to” or “coupled to” another component, it is to be understood that the two components may be directly connected or coupled to each other, or still another component may be interposed therebetween.

In embodiments of the present disclosure, a term such as “module,” “unit,” “part,” or the like is used to refer to a component that performs at least one function or operation, and such a component may be implemented as hardware, software, or a combination thereof. In addition, except for the case where it is necessary to implement each of a plurality of “modules,” “units,” “parts,” or the like as an individual piece of specific hardware, the plurality of “modules,” “units,” “parts,” or the like may be integrated in at least one module or chip and implemented as at least one processor.

Terms like those defined in a commonly used dictionary should be interpreted with meanings consistent with their meanings in the context of the related technology, and should not be interpreted in an ideal or excessively formal sense unless clearly defined in various embodiments of the present disclosure.

Hereinafter, exemplary embodiments will be described in detail with reference to the accompanying drawings.

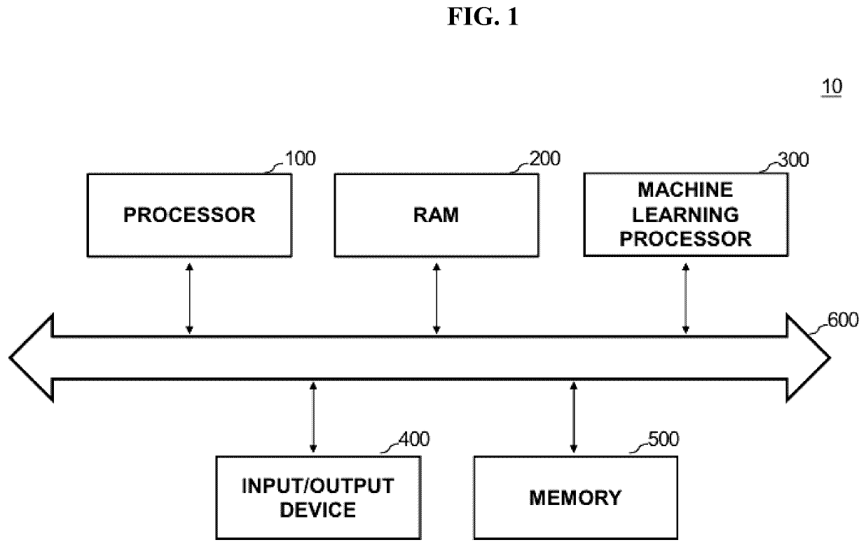

FIG. 1 is a block diagram of an electronic device 10 for generating an integrated report according to an exemplary embodiment of the present disclosure.

Referring to FIG. 1, the electronic device 10 may extract valid information by analyzing input data in real time on the basis of a neural network and analyze a medical image on the basis of the extracted information. For example, the electronic device 10 may be applied to a computing device, a personal computer (PC), a smart television (TV), a smartphone, a mobile device, an image display device, a measurement device, an Internet of things (IoT) device, or the like and may be installed on one of various other kinds of electronic devices.

The electronic device 10 may include at least one Internet protocol (IP) block and a machine learning processor 300. The electronic device 10 may include various kinds of IP blocks. For example, as shown in FIG. 1, the electronic device 10 may include IP blocks such as a processor 100, a random access memory (RAM) 200, an input/output device 400, a memory 500, and the like. In addition, the electronic device 10 may further include general-purpose components such as a multi-format codec (MFC), a video module (e.g., a camera interface, a Joint Photographic Experts Group (JPEG) processor, a video processor, a mixer, or the like), a three-dimensional (3D) graphics core, an audio system, a display driver, a graphics processing unit (GPU), a digital signal processor (DSP), and the like.

The components of the electronic device 10, for example, the processor 100, the RAM 200, the machine learning processor 300, the input/output device 400, and the memory 500, may transmit and receive data through a system bus 600. For example, as a standard bus, an Advanced Microcontroller Bus Architecture (AMBA) protocol of the Advanced Reduced Instruction Set Computer (RISC) Machine (ARM) company or the like may be applied to the system bus 600. However, the system bus 600 is not limited thereto, and various kinds of protocols may be applied.

According to an embodiment, the components of the electronic device 10, that is, the processor 100, the RAM 200, the machine learning processor 300, the input/output device 400, and the memory 500, may be implemented as one semiconductor chip. For example, the electronic device 10 may be implemented as a system on chip (SoC). However, the electronic device 10 is not limited thereto and may be implemented as a plurality of semiconductor chips. According to an embodiment, the electronic device 10 may be implemented as an application processor that is installed in a mobile device.

The processor 100 may control overall operations of the electronic device 10. As an example, the processor 100 may include at least one of a central processing unit (CPU) and a GPU. The processor 100 may include a single core or a plurality of cores. The processor 100 may process or execute programs and/or data stored in the RAM 200 and the memory 500. For example, the processor 100 may control various functions of the electronic device 10 by executing programs stored in the memory 500.

The RAM 200 may temporarily store programs, data, or instructions. For example, programs and/or data stored in the memory 500 may be temporarily loaded into the RAM 200 in accordance with control of the processor 100 or booting code. The RAM 200 may be implemented using a memory such as a dynamic RAM (DRAM), a static RAM (SRAM), or the like.

The input/output device 400 may receive input data from a user or the outside of the electronic device 10 and output a data processing result of the electronic device 10. The input/output device 400 may be implemented using at least one of a touchscreen panel, a keyboard, and various kinds of sensors. According to an embodiment, the input/output device 400 may collect information on the surroundings of the electronic device 10. For example, the input/output device 400 may include at least one of various kinds of sensing devices, such as an imaging device, an image sensor, a light detection and ranging (LIDAR) sensor, an ultrasonic sensor, an infrared sensor, and the like, or may receive a sensing signal from the device. According to an embodiment, the input/output device 400 may detect or receive an image signal from the outside of the electronic device 10 and convert the detected or received image signal into image data, that is, an image frame. The input/output device 400 may store the image frame in the memory 500 or provide the image frame to the machine learning processor 300.

The memory 500 may be a storage for storing data and may store, for example, an operating system (OS), various programs, and various data. The memory 500 may be a DRAM but is not limited thereto. The memory 500 may include at least one of a volatile memory and a non-volatile memory. The non-volatile memory may include a read-only memory (ROM), a programmable ROM (PROM), an electrically programmable ROM (EPROM), an electrically erasable and programmable ROM (EEPROM), a flash memory, a phase-change RAM (PRAM), a magnetic RAM (MRAM), a resistive RAM (RRAM), a ferroelectric RAM (FRAM), and the like. The volatile memory may include a DRAM, an SRAM, a synchronous DRAM (SDRAM), and the like. Also, according to an embodiment, the memory 150 may be implemented as a storage device such as a hard disk drive (HDD), a solid-state drive (SSD), a compact flash (CF) card, a secure digital (SD) card, a micro-SD card, a mini-SD card, an extreme digital (xD) card, a memory stick, or the like.

The machine learning processor 300 may train at least one of various kinds of machine learning models on the basis of previously acquired training data and perform computation on the basis of the trained model. As an example, the machine learning processor 300 may perform computation on the basis of the received input data and generate an inference value or retrain the machine learning model on the basis of the computation result.

The types of machine learning models that are trained and used for inference by the machine learning processor 300 may include a supervised learning model, an unsupervised learning model, and a reinforcement learning model, and a machine learning model may be an ensemble of various kinds of models.

In addition, the machine learning processor 300 may generate a neural network model, train a neural network, or perform computation on the basis of the received input data and generate an information signal or retrain the neural network using the computation result. The neural network may include, but is not limited to, various kinds of neural network models such as a convolutional neural network (CNN), a region with convolutional neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzmann machine (RBM), a fully convolutional network, a long short-term memory (LSTM) network, a classification network, and the like.

The electronic device 10 according to an exemplary embodiment of the present disclosure may perform preprocessing on the input data using the processor 100. The preprocessing of the input data may be a process for effectively processing the collected data for a purpose. As an example, the processor 100 may perform data cleaning, data transformation, data filtering, data integration, and data reduction on a medical image.

The processor 100 of the electronic device 10 according to an exemplary embodiment of the present disclosure may compare a first type report with a second type report and provide an integrated report on the basis of mapping regions corresponding to each other. Here, the machine learning processor 300 may identify additional positions for adding non-mapping regions which are not mapped between a plurality of reports to the integrated report. In addition, the machine learning processor 300 may also identify whether any region in the non-mapping regions may be additionally mapped.

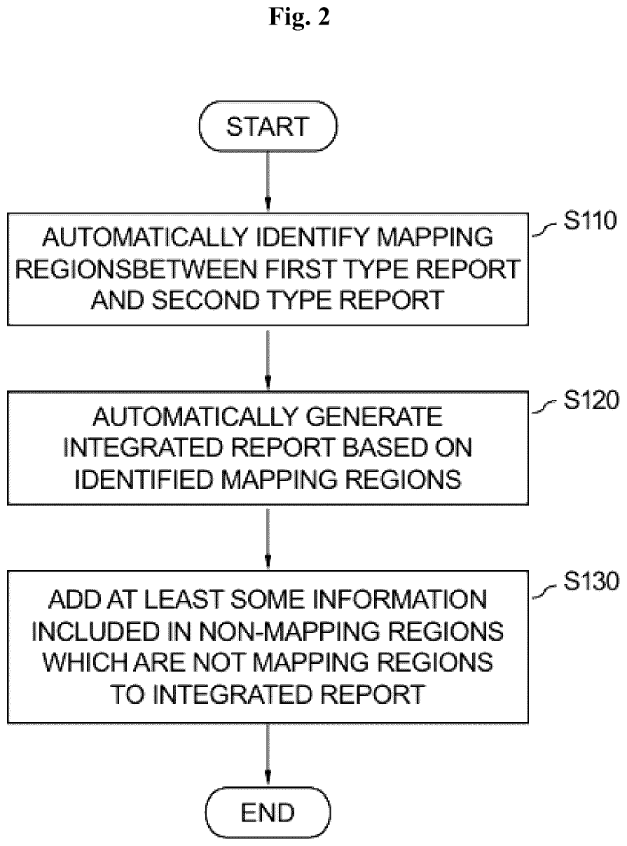

FIG. 2 is a flowchart illustrating a method of generating an integrated report according to an exemplary embodiment.

Referring to FIG. 2, the electronic device 10 according to an exemplary embodiment may automatically generate an integrated report on the basis of identified mapping regions and add at least some information included in non-identified mapping regions to the integrated report. The mapping regions may be regions in which at least some content and identification information included in reports overlap. In other words, the mapping regions may be regions containing the same information in different types of reports. As an example, the mapping regions may include patient identification information (name, age, sex, and the like) and a disease name.

In operation S110, the electronic device 10 may automatically identify mapping regions between a first type report and a second type report. When a user instructs the electronic device 10 on mapping between the reports, the electronic device 10 may automatically identify field identification elements and compare the field identification elements to determine whether the field identification elements are regions to be mapped.

According to an embodiment, when field identification elements have the same value, the electronic device 10 may designate regions corresponding to the field identification elements as mapping regions. When the field identification elements are text, the electronic device 10 may perform natural language processing. When the field identification element included in the first type report and the field identification element included in the second type report are similar text, the electronic device 10 may designate the corresponding regions as mapping regions.

Each report may include a plurality of field identification elements, and the electronic device 10 may identify a plurality of regions on the basis of the field identification elements. In other words, the electronic device 10 may first search for a field identification element in a report and secondly search text surrounding the field identification element for a range of the description of the field identification element.

As an example, when “patient information” is written in the first type report as one field identification element, the electronic device 10 may identify, as one region, a region in which personal information of the patient is written around the text “patient information.” Likewise, the electronic device 10 may identify a region around the field identification element of “patient information” in the second type report.

Field identification elements may be item information about the content of a report such as patient information, a disease name, a lesion location, and the like. However, embodiments of the present disclosure are not limited thereto, and a field identification element may be an element indicating what kind of information each region in the content of a report is about.

In operation S120, the electronic device 10 may automatically generate an integrated report on the basis of the identified mapping regions. The electronic device 10 may merge the content of the mapping regions corresponding to each other in the first type report and the second type report. Here, content that is not included in both of the reports in common may be added to the integrated report.

In operation S130, the electronic device 10 may add at least some information included in non-mapping regions which are not the mapping regions included in the integrated report to the integrated report. The non-mapping regions may be regions that are not included in the integrated report in operation S120.

Here, the electronic device 10 may determine a relationship between a field identification element of a non-mapping region and a field identification element of a mapping region in each type of report and identify a position to which the non-mapping region will be added in the integrated report on the basis of the relationship. The electronic device 10 may identify a hierarchical relationship between field identification elements in each type of report and determine which position a non-mapping region will be added to in the integrated report on the basis of the hierarchical relationship.

As an example, the electronic device 10 may identify a first field identification element to be in a higher layer level than a second field identification element, and when a region corresponding to the first field identification element is identified as a mapping region and a region corresponding to the second field identification element is identified as a non-mapping region, may add content corresponding to the second field identification element under the first field identification element in the integrated report.

The electronic device 10 may designate a position to which information of a non-mapping region will be added using a non-mapping data addition model with reference to not only a method of identifying a position to which a non-mapping region will be added in hierarchical relationships between field identification elements but also a previous integrated report generation history.

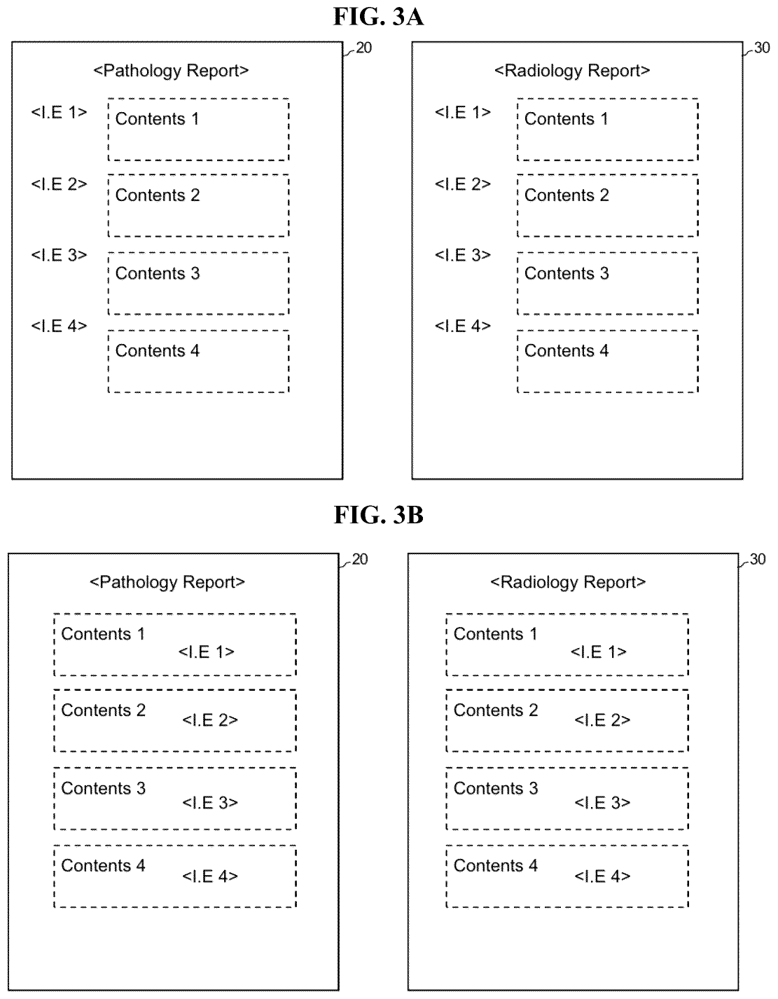

FIGS. 3A and 3B are diagrams of different types of reports according to an exemplary embodiment.

Referring to FIGS. 3A and 3B, a first type report 20 may be a pathological report, and a second type report 30 may be a radiological report. The pathological report may be a report describing the results of analyzing a whole slide image which is obtained by taking a piece of tissue from a patient's lesion site and magnifying the piece of tissue down to the cellular level. The radiological report may be a report describing the results of analyzing cross-sectional body image information acquired through radiology such as X-ray, computed tomography (CT), magnetic resonance imaging (MRI), or the like.

The pathological report is results of analyzing the patient's lesion through an invasive test, and the radiological report is results of analyzing the patient's lesion through a non-invasive test. Information with different characteristics is acquired from the two kinds of reports. The pathological report and the radiological report are analyzed by medical workers with different expertise, and a very small number of medical workers may analyze both the pathological and radiological images to write a single integrated report.

Therefore, the demand to see the results of pathological and radiological image analysis at once is continuously growing, but the results are not currently being integrated and provided as a single report. Accordingly, an integrated report provision method of the present disclosure can provide a method of merging reports generated by experts in different fields.

FIGS. 3A and 3B illustrate a method of generating an integrated report on the basis of a pathological report and a radiological report, but types of reports integrated according to an embodiment of the present disclosure are not limited thereto.

Referring to the reports according to the embodiment of FIG. 3A, field identification elements may be written on the left side of each report, and the content of each field identification element may be written. First content may be written about a first field identification element on the first type report 20, and the electronic device 10 may identify all content preceding a second field identification element as a region corresponding to the first field identification element.

Likewise, first content may be written about a first field identification element on the second type report 30, and the electronic device 10 may identify all content preceding a second field identification element as a region corresponding to the first field identification element.

The electronic device 10 may determine whether each field identification element of the first type report 20 corresponds to each field identification element of the second type report 30, and designate regions corresponding to field identification elements corresponding to each other as mapping regions. The electronic device 10 may generate an integrated report on the basis of the mapped regions and add at least a part of a non-mapping region that is not designated as a mapping region to the integrated report.

Referring to the reports according to the embodiment of FIG. 3B, field identification elements may be included in the content of the reports. The electronic device 10 may identify keywords which are predetermined as field identification elements in each report and divide regions centering on the field identification elements. As an example, the electronic device 10 may designate a region for each paragraph including a field identification element.

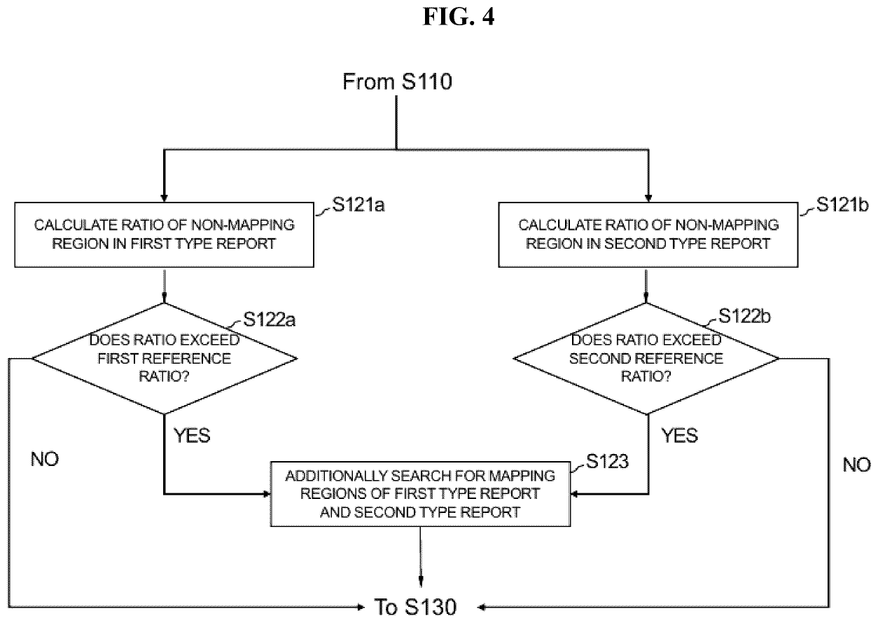

FIG. 4 is a flowchart illustrating a process of additionally searching the first type report 20 and the second type report 30 for mapping regions according to an exemplary embodiment.

Referring to FIG. 4, the electronic device 10 according to an exemplary embodiment may acquire mapping regions between the first type report 20 and the second type report 30 and then additionally search for mapping regions on the basis of a ratio of a non-mapping region which is not mapped. The electronic device 10 may comparatively map the two different types of reports on the basis of field identification elements, but when a ratio of a non-mapping region is larger than a reference ratio, may determine whether any content of the non-mapping region may be additionally mapped.

In operation S121a, the electronic device 10 may calculate a ratio of a non-mapping region in the first type report 20. The ratio of the non-mapping region may be a ratio of text corresponding to the non-mapping region to all the text of the first type report 20.

In operation S122a, the electronic device 10 may determine whether the ratio of the non-mapping region in the first type report 20 exceeds a first reference ratio. When the ratio exceeds the first reference ratio, the electronic device 10 may additionally search for mapping regions of the first type report 20 and the second type report 30.

In operation S121b, the electronic device 10 may calculate a ratio of a non-mapping region in the second type report 30. The ratio of the non-mapping region may be a ratio of text corresponding to the non-mapping region to all the text of the second type report 30.

In operation S122b, the electronic device 10 may determine whether the ratio of the non-mapping region in the second type report 30 exceeds a second reference ratio. When the ratio exceeds the second reference ratio, the electronic device 10 may additionally search for mapping regions of the first type report 20 and the second type report 30.

When the ratio of the non-mapping region in the first type report 20 is the first reference ratio or less and the ratio of the non-mapping region in the second type report 30 is the second reference ratio or less, the electronic device 10 may perform operation S130 according to the exemplary embodiment of FIG. 2. On the other hand, in operation S123, when any one of the ratios exceeds the corresponding reference ratio, the electronic device 10 may additionally search for mapping regions of the first type report 20 and the second type report 30.

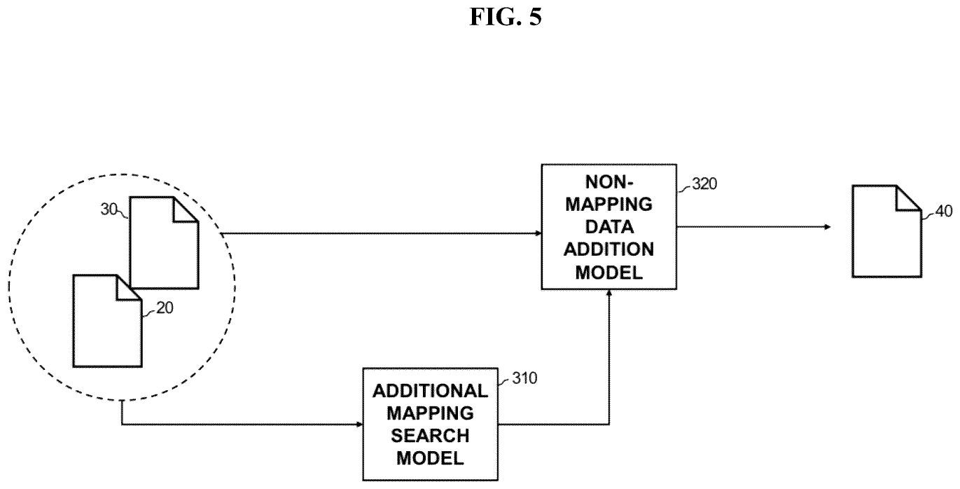

An additional search for mapping areas will be described below with reference to FIG. 5.

FIG. 5 is a diagram of a process in which an integrated report 40 is generated by a plurality of trained models according to an exemplary embodiment.

Referring to FIG. 5, the electronic device 10 may first specify mapping regions between the first type report 20 and the second type report 30 and then additionally specify mapping regions and add a non-mapping region to the integrated report 40 on the basis of an additional mapping search model 310 and/or a non-mapping data addition model 320.

The additional mapping search model 310 and the non-mapping data addition model 320 may be neural network models that are trained in advance. The additional mapping search model 310 may be a model for additionally searching the first type report 20 and the second type report 30 for mapping regions, and the non-mapping data addition model 320 may be a model for identifying a position to which a non-mapping region will be added in the integrated report 40.

According to an embodiment, when the ratio of a non-mapping region in each of the first type report 20 and the second type report 30 exceeds a corresponding reference ratio, the electronic device 10 may input the first type report 20 and the second type report 30 to the additional mapping search model 310. The electronic device 10 may acquire transformed identification elements as an output result of the additional mapping search model 310, remap the first type report 20 and the second type report 30 to each other, and then input the first type report 20 and the second type report 30 to the non-mapping data addition model 320.

When the ratio of the non-mapping region in each of the first type report 20 and the second type report 30 is the corresponding reference ratio or less, the electronic device 10 may directly input the first type report 20 and the second type report 30 to the non-mapping data addition model 320 without inputting the two reports to the additional mapping search model 310.

According to an embodiment, the electronic device 10 may search a database for relation reports corresponding to a non-mapping region and output the relation reports, and the additional mapping search model 310 may receive the relation reports and transformation histories of the relation reports and acquire candidate identification elements for the non-mapping region. The relation reports may be the same type of reports in which a field identification element included in the non-mapping region is written.

The additional mapping search model 310 is a generative artificial intelligence (AI) model that learns field identification element transformation patterns of relation reports. When the first type report 20 and the second type report 30 are received, the additional mapping search model 310 may output the candidate identification elements corresponding to the field identification element of the non-mapping region.

The electronic device 10 acquires the candidate identification elements from the additional mapping search model 310 and then matches first candidate identification elements for the first type report 20 and second candidate identification elements for the second type report 30 with each other. The electronic device 10 may compare the content of matched candidate identification elements among the first candidate identification elements and the second candidate identification elements.

The electronic device 10 may store an ontology database in which semantic connections of concepts corresponding to types of reports are defined, and compare content on the basis of the ontology database. As an example, the relationships between content keywords associated with the matched first and second candidate identification elements may be acquired from the ontology database, and a ratio of associated keywords to all keywords may be calculated as a content matching degree.

When the content matching degree is a reference matching degree or higher, the electronic device 10 may set a first candidate identification element as a transformation identification element for the first type report 20 and set a second candidate identification element as a transformation identification element for the second type report 30.

In the same manner as described above, the electronic device 10 of the present disclosure may determine whether any field identification elements in the non-mapping regions of the first type report 20 and the second type report 30 may be additionally mapped, and then map field identification elements.

When the ratio of the non-mapping region in each of the first type report 20 and the second type report 30 is the corresponding reference ratio or less because the additional mapping search model 310 additionally designates a mapping region in the first type report 20 and the second type report 30, the electronic device 10 may add data of the non-mapping regions to the integrated report 40.

The non-mapping data addition model 320 is a model that learns relationships between field identification elements, and may infer relationships between field identification elements of a non-mapping region and a mapping region in each type report. The relationships between the field identification elements may be quantified data based on correlations and hierarchical relationships.

The electronic device 10 may input the first type report 20 and the second type report 30 to the non-mapping data addition model 320 and identify a position to which the non-mapping region will be added in the integrated report 40 on the basis of the inferred relationship between field identification elements. As an example, the electronic device 10 may search the non-mapping regions of the first type report 20 and the second type report 30 for field identification elements that are highly relevant to a first field identification element which is set as a mapping region and included in the integrated report 40. The electronic device 10 may include the field identification elements that are determined to be highly relevant in sub-items of the first field identification element.

According to an embodiment, when a plurality of field identification elements of a non-mapping region are identified to be highly relevant to a field identification element of a mapping region, the electronic device 10 may determine the order of field identification elements in the integrated report 40 on the basis of the correlations in content corresponding to the field identification elements.

As an example, the electronic device 10 may identify a second field identification element and a third field identification element of a non-mapping region that are highly relevant to the first field identification element of the mapping region. Here, the electronic device 10 may extract content keywords associated with each field identification element and calculate content matching degrees between first content keywords corresponding to the first field identification element and second content keywords corresponding to the second field identification element. Likewise, the electronic device 10 may calculate content matching degrees between the first content keywords and third content keywords corresponding to a third field identification element.

The electronic device 10 may determine whether semantic connections are defined between the concepts of keywords by the ontology database and calculate a content matching degree in proportion to the number of keywords between which connections are defined. When a content matching degree of the second content keywords is higher than a content matching degree of the third content keywords, the electronic device 10 may place the second field identification elements prior to the third field identification elements in the integrated report 40.

Meanwhile, methods according to the above-described various embodiments of the present invention may be implemented in the form of an application or a software program that can be installed on an existing electronic device.

Also, the methods, in whole or in part, may be configured as software function modules and implemented on an OS. Otherwise, each step may be configured as one software function module and implemented on the OS. Therefore, although some embodiments of the present disclosure are not implemented in their entirety by one software function module, when several software function modules implement each step of the present invention and run on one OS, it is to be understood that the method of the present disclosure is implemented.

Also, methods according to the above-described various embodiments of the present invention may be simply implemented through a software upgrade or hardware upgrade of an existing electronic device. In addition, the above-described various embodiments of the present invention can be implemented through an embedded server provided in the electronic device or an external server of the electronic device.

Meanwhile, according to an embodiment of the present invention, the above-described various embodiments may be implemented as software including instructions stored in a recording medium that is readable by a computer or a similar device thereto using software, hardware, or a combination thereof. In some cases, the embodiments described in the present specification may be implemented as a processor itself. According to software implementation, the embodiments, such as procedures and functions, described in the present specification may be implemented as separate software modules. Each of the software modules may perform one or more of the functions and operations described in the present specification.

Meanwhile, the computer or the similar device thereto is a device that can call a stored instruction from a storage medium and operate in accordance with the called instruction, and may include a device according to the disclosed embodiments. When the instruction is executed by a processor, the processor may perform a function corresponding to the instruction by itself or using other components under control of the processor. The instruction may include code that is generated or executed by a compiler or an interpreter.

A machine-readable recording medium may be provided in the form of a non-transitory computer-readable recording medium. Here, the non-transitory computer-readable recording medium is a storage medium that does not include any signal and is tangible irrespective of whether data is stored in the storage medium semi-permanently or temporarily. The non-transitory computer-readable recording medium is a medium that stores data semi-permanently rather than storing data for a very short time, such as a register, a cache, a memory, or the like, and is readable by a machine. Examples of the non-transitory computer-readable recording medium may be a compact disc (CD), a digital versatile disc (DVD), an HDD, a Blu-ray disc, a Universal Serial Bus (USB) memory, a memory card, a ROM, and the like.

In an integrated report according to an exemplary embodiment of the present disclosure, information that is analyzed in different reports regarding the same disease of the same patient is gathered as a single report, and thus it is possible to provide a method for a patient and medical worker to easily identify the patient's state. In addition, an integrated report of the present disclosure allows information processing for not only mapping regions corresponding to each other in reports written in different formats but also non-mapping regions which are not mapped, and thus it is possible to provide a method of merging a plurality of reports without loss.

Effects that can be achieved from exemplary embodiments of the present disclosure are not limited to those described above, and other effects that have not been described will be clearly derived and understood by those of ordinary skill in the art from the above description. In other words, unintended effects of practicing the exemplary embodiments of the present disclosure may also be derived from the exemplary embodiments of the present disclosure by those of ordinary skill in the art.

Exemplary embodiments have been disclosed above in the drawings and specification. While specific terms have been used to describe embodiments herein, the terms are only used to describe the technical spirit of the present disclosure and are not intended to limit the meaning or scope of the present disclosure stated in the claims. Therefore, those of ordinary skill in the art should understand that various modifications and other equivalent embodiments are possible from the embodiments. Accordingly, the technical scope of the present disclosure will be determined from the technical spirit of the following claims.

What is claimed is:

1. A method of providing an integrated report for enterprise healthcare data, the method comprising:
automatically identifying mapping regions between a first type report and a second type report different from the first type report;
automatically generating an integrated report on the basis of the identified mapping regions; and
adding at least some information included in non-mapping regions, which are regions of the first type report and the second type report other than the mapping regions, to the integrated report.

2. The method of claim 1, wherein the adding of the at least some the information to the integrated report comprises:
determining relationships between field identification elements of the non-mapping regions and field identification elements of the mapping regions; and
identifying positions to which the non-mapping regions will be added in the integrated report on the basis of the relationships.

3. The method of claim 2, wherein the determining of the relationship comprises determining correlations between the non-mapping regions and the mapping regions on the basis of a previously generated integrated report.

4. The method of claim 2, further comprising outputting a relation report corresponding to the non-mapping regions when the non-mapping regions in each of the first type report and the second type report exceed a reference ratio.

5. The method of claim 4, further comprising additionally searching the first type report and the second type report for mapping regions on the basis of the relation report.

6. The method of claim 5, wherein the outputting of the relation report comprises transforming field identification elements of the non-mapping regions of each of the first type report and the second type report on the basis of the relation report.

7. The method of claim 6, wherein the transforming of the field identification elements comprises:
acquiring candidate identification elements for the non-mapping regions on the basis of a plurality of relation reports;
matching first candidate identification elements for the first type report and second candidate identification elements for the second type report;
calculating a content matching degree of the matched first and second candidate identification elements; and
transforming the field identification elements of the non-mapping regions on the basis of the content matching degree.
